SEQUENCE LISTING (19 sequences)

SEQ ID NO: 1 - protein, 351 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 381 aa, homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 382 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 410 aa, homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 17 aa, Homo sapiens
AEPRQEFEVMEDHAGTY

SEQ ID NO: 8 - protein, 11 aa, Human immunodeficiency virus
YGRKKRRQRRR

SEQ ID NO: 9 - protein, 10 aa, Homo sapiens
MIIYRDLISH

SEQ ID NO: 10 - protein, 14 aa, Drosophila melanogaster
RQIKIWFQNRRMKW

SEQ ID NO: 11 - protein, 18 aa, Artificial Sequence
  synthetic
KLALKLALKALKAALKLA

SEQ ID NO: 12 - protein, 27 aa, Homo sapiens
GWTLNSAGYLLGKINLKALAALAKKIL

SEQ ID NO: 13 - protein, 27 aa, Simian virus 40
GALFLGWLGAAGSTMGAWSQPKKKRKV

SEQ ID NO: 14 - protein, 18 aa, Homo sapiens
RGGRLSYSRRRFSTSTGR

SEQ ID NO: 15 - protein, 10 aa, Homo sapiens
RRLSYSRRRF

SEQ ID NO: 16 - protein, 13 aa, Artificial Sequence
  synthetic
AKFVAAWTLKAAA

SEQ ID NO: 17 - protein, 37 aa, Homo sapiens
AKAKTDHGAEIVYKSPVVSGDTSPRHLSNVSSTGSID

SEQ ID NO: 18 - protein, 776 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 19 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

(19) United States
(12) Patent Application Publication
(10) Pub. No.: US 2014/0056901 A1
(43) Pub. Date: Feb. 27, 2014

(54) ANTI-TAU ANTIBODIES AND COMPOSITIONS FOR AND METHODS OF MAKING AND USING IN TREATMENT, DIAGNOSIS AND MONITORING OF TAUOPATHIES

(71) Applicant: THE INSTITUTE FOR MOLECULAR MEDICINE, South Laguna Beach, CA (US)

(72) Inventors: Michael G. Agadjanyan, Huntington Beach, CA (US); Anahit Ghochikyan, Huntington Beach, CA (US)

(21) Appl. No.: 13/839,135

(22) Filed: Mar. 15, 2013

Related U.S. Application Data
(60) Provisional application No. 61/691,607, filed on Aug. 21, 2012.
(60) Provisional application No. 61/759,216, filed on Jan. 31, 2013.
(60) Provisional application No. 61/763,358, filed on Feb. 11, 2013.

(51) Int. Cl. C07K 16/18

(52) U.S. Cl. CPC C07K 16/18
USPC 424/139.1; 424/184.1; 424/193.1; 435/331; 514/44 R; 530/350; 530/387.3; 530/387.9; 536/23.53

(57) ABSTRACT

The disclosure provides antibodies that bind the N-terminal region of tau and also bind to pathological tau aggregates, conformational epitopes and peptides mimicking these epitopes (mimotopes). The antibodies may be used to treat tauopathies (e.g. Alzheimer's disease).

### RELATED APPLICATION DATA

This application claims the benefit of U.S. 61/691607, filed 21 Aug. 2012, and U.S. 61/759,216, filed 31 Jan. 2013 and U.S. 61/763,358, filed 11 Feb. 2013.

### STATEMENT REGARDING SEQUENCE LISTING

The sequence listing associated with this application is provided in text format in lieu of a paper copy, and is hereby incorporated by reference into the specification. The name of the text file containing the sequence listing is NI201_seq-list.txt; the text file is 36,681 bytes, was created on 15 Mar. 2013, and is being submitted electronically via EFS-web. A substitute sequence listing was created on 7 Jun. 2013; the name of the text file containing this sequence listing is NI201_seq-list-corrected.txt; the text file is 36,831 bytes and was submitted electronically via EFS-web. It is hereby incorporated by reference in its entirety into the specification.

### TECHNICAL FIELD

This patent application relates generally to antibodies that react with tau and methods using these antibodies in treatment of tauopathies, including Alzheimer's Disease.

### BACKGROUND

The misfolding and aggregation of some specific proteins is a hallmark of a variety of neurodegenerative disorders, including but not limited to Alzheimer's Disease (AD) and frontotemporal dementia. Much attention and research has focused on deposition of amyloid-β(Aβ) in senile plaques, although aggregation of pathological tau protein in neurofibrillary tangles also plays an important role in disease progression.

Tau is a microtubule-associated protein. In AD, tau undergoes several changes to a pathological state. Tau can be abnormally folded and phosphorylated resulting in the generation of neurofibrillary tangles toxic to neurons. In AD, amyloid accumulation in the brain can occur decades before the beginning of symptoms such as memory loss and personality change. Current data suggest that Aβ pathology emerges prior to tau pathology, but may accelerate toxic neurofibrillary tangle formation. At best however, anti-Aβ immunotherapy only slightly decreases tau pathology and often does not affect the level of pathological tau at all. Moreover, pathological tau burden in the brains of patients with mild to moderate AD plays an important role in disease progression.

### SUMMARY

The disclosure is directed to polyclonal, monoclonal and other forms of antibodies that recognize and bind to pathological forms of tau protein. Antibodies include fragments comprising the binding site, single-chain antibodies, humanized antibodies, and the like. In other aspects, epitopes of the antibodies capable of recognizing a pathogenic conformation of prefibrillar pathological or neurotoxic tau and its precursors are provided and may be used as immunogens. Hybridomas that produce monoclonal antibodies capable of binding to pathological neurotoxic forms of tau and its precursors are provided.

The disclosure is also directed to an antibody capable of recognizing a pathogenic conformation of prefibrillar pathological or neurotoxic tau and its precursors. In another aspect, antibodies recognizing peptides mimicking the aggregated tau (mimotopes) are provided. The epitopes and mimotopes may be used as antigens in immunization.

Other aspects are directed to pharmaceutical compositions (e.g. antibodies) for use in the treatment of and/or prevention of tauopathies. Methods are also provided for preparing and using (i) antibodies capable of recognizing a N-terminus region of tau and a pathological neurotoxic forms of tau and its precursors for the diagnosis of and for therapeutic intervention in tauopathies (e.g. Alzheimer's disease); (ii) antibodies capable of recognizing mimotope(s) of tau molecules for the diagnosis of and/or for therapeutic intervention in tauopathies (e.g. Alzheimer's disease).

Methods of diagnosing, monitoring and treating tauopathies are provided.

These and other aspects of the present invention will become evident upon reference to the following detailed description and attached drawings.

### BRIEF DESCRIPTION OF THE DRAWINGS

FIGS. 1A-C show humoral (A, B) and cellular (C) responses in B6SJL immunized with tau₂₋₁₈ fused with P30 peptide from tetanus toxoid. Mice were immunized biweekly (3 times) with 100 μg/mouse tau₂₋₁₈-P30 vaccine formulated in Quil-A adjuvant. Control mice were immunized with an irrelevant peptide in Quil-A. (A) Titers of antibody specific to tau₂₋₁₈ peptide were determined in serially diluted individual sera. Lines indicate the average of mice (n=6 for experimental group) and n=4 for control group. (B) Antibodies specific to three different forms of tau proteins: wild/type (4R/0N) tau, P301L and mutated form (Δ19-29) of 4R0N proteins were detected in immune sera diluted 1:600. Lines indicate the average of OD₄₅₀ (n=6). (C) IFN-γ producing cells were detected in the cultures of immune splenocytes activated with tau₂₋₁₈-P30 or P30 peptides, but not tau₂₋₁₈ itself. The number of IFNγ producing splenocytes was analyzed by ELISPOT assay after ex vivo re-stimulation of cells with 10 μg/ml tau₂₋₁₈ and P30 peptides (C) or tau₂₋₁₈-P30 peptide (data not shown). Error bars indicate average±s.d. (n=4; P≦001).

FIGS. 2A-C illustrate humoral (ELISA and Dot Blot assay) and cellular (ELISPOT assay) immune responses after immunization with tau₃₈₂₋₄₁₈. Titers of anti-tau₃₈₂₋₄₁₈-specific antibodies against various tau molecules (panel A); Immunostaining of fibrillar tau molecules (panel B). T cell response is shown in panel C.

FIG. 3 shows photographs of brain sections stained with anti-tau₂₋₁₈ antibody. Sera from mice immunized with tau₂₋₁₈-P30 and control antisera from mice immunized with an irrelevant antigen were used to stain sections of Alzheimer's Disease (AD) brain and non-AD brain. Original magnifications: 10×, scale bar=100 μm; 20×, scale bar=50 μm.

FIGS. 4A-4B show (A) a graph of FRET analysis of anti-tau₂₋₁₈ antibody used to block the ability of brain lysate from transgenic mice to induce aggregation of intracellular repeat domain (RD). Brain lysate was either untreated or treated with anti-tau₂₋₁₈ antibody and added to HEK293 cells co-transfected with RD(ΔK)-CFP/YFP prior to FRET analysis. Increased FRET signal was detected in wells with untreated brain lysate. Treatment of lysate with anti-tau₂₋₁₈ antibody decreased FRET signal due to blocking the full-length tau in brain lysate and inhibition of induction of RD aggregation. FIG. 4B presents confocal microscope images of exemplary binding of anti-tau₂₋₁₈ antibody to brain lysate. (Panel A) Non-transfected HEK293 cells stained with anti-tau₂₋₁₈ antibody followed by secondary anti-mouse Ig conjugated with Alexa546. (Panel B) Non-transfected HEK293 cells stained with brain lysate/anti-tau₂₋₁₈ complexes followed by secondary anti-mouse immunoglobulin conjugated with Alexa546. (Panel C) RD-YFP-transfected HEK293 cells stained with brain lysate/anti-tau₂₋₁₈ complexes followed by secondary anti-mouse immunoglobulin conjugated with Alexa546. (Panel D) YFP-transfected HEK293 cells stained with anti-tau₂₋₁₈ antibody followed by secondary anti-mouse immunoglobulin conjugated with Alexa546.

FIGS. 5A and B show efficacy of anti-tau₂₋₁₈ antibody detected in ex vivo model system. Figure (A) demonstrates inhibition of trans-cellular propagation of tau aggregates by anti-tau₂₋₁₈ antibody. HEK293 cells transfected with RD(LM)-HA were co-cultured for 48 h with an equivalent number of HEK293f cells co-transfected with RD(ΔK)-CFP/YFP prior to FRET analysis. Increased FRET signal was detected in co-cultured cells. Addition of serial dilutions of purified anti-tau₂₋₁₈ antibody decreased FRET signal due to inhibition of trans-cellular propagation of aggregated RD. Figure (B) demonstrates the binding of anti-tau₂₋₁₈ antibodies to RD-(ΔK)YFP aggregates. HEK293 cells were transfected with RD(ΔK)-YFP or were mock-transfected (NT). Anti-tau₂₋₁₈ antibody was added to the culture medium for 48 h. Cells were fixed, permeabilized, and stained with an anti-mouse secondary antibody labeled with Alexa 546 and analyzed by confocal microscopy. Anti-tau₂₋₁₈/RDΔ(K)-YFP complexes were identified when RDΔ(K)-YFP is expressed but not in its absence (NT).

### DETAILED DESCRIPTION

The present disclosure provides polyclonal, monoclonal, antibody fragments, single-chain antibodies, and other forms of antibodies specific to pathological tau aggregates, immunogenic tau peptides and tau epitopes or mimotopes recognized by these antibodies, hydridomas producing these antibodies, uses of the antibodies, and immunogenic tau peptides in preparation of pharmaceutical compositions for the treatment of tauopathies (e.g. Alzheimer's disease), and uses of these antibodies and their pharmaceutical compositions in the treatment of tauopathies, and uses of the antibodies for diagnosis and monitoring of tauopathies.

1. Anti-Tau Antibodies and Tau Protein

Tau protein is used as an antigen to generate an immune response. Tau is a microtubule-associated protein found primarily in neurons and glia, but also in other areas of the CNS. Six isoforms have been identified, primarily differing by their number of binding domains. The isoforms result from alternative splicing. Three isoforms have three binding domains (3R), and three have four (4R). Exons 2 and 3 are variably present. In two isoforms, both are present (2N), in two other isoforms, just exon 3 is present (1N), and in two, neither are present (0N). The binding domains of tau are located in the C-terminus region. The six isoforms are called 0N3R (SEQ ID NO.1), 0N4R (SEQ ID NO.2), 1N3R (SEQ ID NO.3), 1N4R (SEQ ID NO.4), 2N3R (SEQ ID NO.5), and 2N4R (SEQ ID NO. 6). In addition, tau may be phosphorylated. Aggregation of tau proteins is a common feature of numerous neurodegenerative disorders.

The N-terminal region of tau is normally found interior due to folding of the protein, and it is exposed during aggregation of tau (Morfini G A, 2009, J Neurosciences; Horowitz P M, 2004, JN Neurosciences). It is also termed as phosphatase-activating domain (PAD) and plays a role in activation of a signalling cascade involving protein phosphatase I and glycogen synthase kinase 3, which leads to anterograde FAT inhibition. The C-terminal region of tau is also normally not exposed on the surface of the protein. The N-terminal region may be used to generate antibodies to tau. The N-terminal region may be from residues about 1 to about 100, from about 1 to about 50, from about 1 to about 25, from about 1 to about 20, from 1 to about 15. In certain embodiments, the region starts at residue 2. In other embodiments, the region is from residue 2 through 18 and comprises the sequence AEPRQEFEVMEDHAGTY (SEQ ID NO.7), called tau₂₋₁₈. The C-terminal region of tau is also normally not exposed on the surface of the protein and may be used to generate antibodies to tau. The C-terminal region is common to all the isoforms and comprises the last about 100 residues. A peptide from this region, or from about the last 75 residues, or from about the last 50 residues, or from about the last 25 residues may be used to generate anti-tau antibodies. An exemplary peptide is AKAKTDHGAEIVYKSPVVSGDTSPRHLSNVSSTGSID (SEQ ID NO. 17). The region used for the generation of anti-tau antibodies is referred to herein as “tau epitope”. It is preferable that the tau epitope be non-phosphorylated. Other regions may also be used to generate anti-tau therapeutic antibodies. (See example 4).

A tau epitope should be able to induce a humoral immune response. Verification that a candidate sequence induces a humoral response can be performed by synthesizing the sequence and coupling it to a carrier protein that is used to immunize an animal, e.g. a mouse. Sera may then be tested by ELISA or other known method for the presence of antibodies to the candidate. In addition, the epitopes may be tested by any method known in the art or described herein for stimulation of T cells. Suitable epitopes do not stimulate T cells.

Depending on size and immunogenicity, among other factors, the tau epitope may be coupled to a carrier molecule, typically a protein. Carrier proteins are well-known in the art and include tetanus toxin, diphtheria toxoid, Hepatitis B surface antigen, influenza virus hemagglutinin, influenza virus matrix protein, serum albumin, and the like. In some embodiments, fragments of the carrier proteins are used.

2. Antibodies to Tau

Antibodies to tau are provided herein. Antibodies are raised to the N-terminal region of tau, and in certain embodiments, antibodies are raised to tau₂₋₁₈. Antibodies should bind an epitope in the N-terminal region of tau. In some embodiments, the antibodies are capable of binding to the repeat domain (RD) of tau, to aggregated RD domains, to recombinant human tau; to pathologically modified tau; to pathologically aggregated tau at the pre-tangle stage, in neurofibrillary tangles (NFT), neuropil threads and dystrophic neurites in the brain; to any of the six tau isoforms; to amino acids 2-18 of tau; to a conformational antigenic determinant that occurs in the pathological form of tau; and to a peptide mimicking (mimotope) the tau conformational antigenic determinant.

Antibodies that bind pathological tau, but not normal tau are highly desirable, although in general, anti-tau antibodies are not internalized in cells where they could bind functionally normal tau molecules. Antibodies may be used for a variety of purposes, including isolation of tau and inhibiting (antagonist) activity of tau, especially pathological forms of tau. As well, assays for small molecules that interact with pathological tau will be facilitated by the development of antibodies.

Within the context of the present invention, antibodies are understood to include monoclonal antibodies, polyclonal antibodies, anti-idiotypic antibodies, single chain antibodies, antibody fragments (e.g., Fab′, Fab, and F(ab′)2, Fv variable regions, single chain Fv, or complementarity determining regions). Antibodies are generally accepted as specific against tau protein if they bind with a Kd equal to or greater than 10⁻⁷M, preferably equal to or greater than 10⁻⁸M. The affinity of an antibody or binding partner can be readily determined by one of ordinary skill in the art (see Scatchard, Ann. N.Y. Acad. Sci. 51:660-672, 1949).

Briefly, a polyclonal antibody preparation may be readily generated in animals, such as a variety of warm-blooded animals including animals such as monkeys, rabbits, mice, and rats. Typically, mammals are immunized with one of the compositions described herein. Routes of administration include intraperitoneal, intramuscular, intraocular, or subcutaneous injections, usually in an adjuvant (e.g., alum, Freund's complete adjuvant). Polyclonal antisera that bind tau in an assay at least three times greater than background are desirable.

Monoclonal antibodies may also be readily generated from hybridoma cell lines using conventional techniques (see U.S. Pat. Nos. RE 32,011, 4,902,614, 4,543,439, and 4,411,993; see also Antibodies: A Laboratory Manual, Harlow and Lane (eds.), Cold Spring Harbor Laboratory Press, 1988; all references incorporated in their entirety). Briefly, within one embodiment, a subject animal such as a rat or mouse is injected with one of the compositions taught herein. Protein or nucleic acid constructs are typically administered as an emulsion in an adjuvant such as Freund's complete or incomplete adjuvant in order to increase the immune response. Between one and three weeks after the initial immunization, the animal is generally boosted and may tested for reactivity to tau utilizing well-known assays. The spleen and/or lymph nodes are harvested and cells immortalized. Various immortalization techniques, such as mediated by Epstein-Barr virus or fusion to produce a hybridoma, may be used. In one embodiment, immortalization occurs by fusion with a suitable myeloma cell line to create a hybridoma that secretes monoclonal antibody. Suitable myeloma lines include, for example, NS-1 (ATCC No. TIB 18), and P3X63 -Ag 8.653 (ATCC No. CRL 1580). Preferred fusion partners do not express endogenous antibody genes. Following fusion, the cells are cultured in medium containing a reagent that selectively allows for the growth of fused cells. After about seven days, hybridomas may be screened for the presence of antibodies that are reactive against a tau protein. A wide variety of assays may be utilized, including for example countercurrent immuno-electrophoresis, radioimmunoassays, radioimmunoprecipitations, enzyme-linked immuno-sorbent assays (ELISA), dot blot assays, western blots, immunoprecipitation, inhibition or competition assays, and sandwich assays (see U.S. Pat. Nos. 4,376,110 and 4,486,530, incorporated in their entirety; see also Antibodies: A Laboratory Manual, Harlow and Lane (eds.), Cold Spring Harbor Laboratory Press, 1988).

Other techniques may also be utilized to construct monoclonal antibodies (see Huse et al., Science 246:1275-1281, 1989; Sastry et al., Proc. Natl. Acad. Sci. USA 86:5728-5732, 1989; Alting-Mees et al., Strategies in Molecular Biology 3:1-9, 1990; incorporated for their description of recombinant techniques). Briefly, mRNA is isolated from a B cell population and utilized to create heavy and light chain immunoglobulin cDNA expression libraries in suitable vectors, such as *ImmunoZap(H) and *ImmunoZap(L). These vectors may be screened individually or co-expressed to form Fab fragments or antibodies (see Huse et al., supra; Sastry et al., supra). Positive plaques may subsequently be converted to a non-lytic plasmid that allows high level expression of monoclonal antibody fragments from E. coli.

Similarly, portions or fragments, such as Fab and Fv fragments, of antibodies that contain the antigen-binding site may also be constructed utilizing conventional enzymatic digestion or recombinant DNA techniques to yield isolated variable regions of an antibody. In one embodiment, the genes which encode the variable region from a hybridoma producing a monoclonal antibody of interest are amplified using nucleotide primers for the variable region. These primers may be synthesized by one of ordinary skill in the art, or may be purchased from commercially available sources (e.g., Stratacyte, La Jolla, Calif.) Amplification products are inserted into vectors such as ImmunoZAP™ H or ImmunoZAP™ L (Stratacyte), which are then introduced into E. coli, yeast, insect cells, or mammalian-based systems for expression. Utilizing these techniques, large amounts of a single-chain protein containing a fusion of the VH and VL domains (scFv) may be produced (see Bird et al., Science 242:423-426, 1988, incorporated in its entirely). In addition, techniques may be utilized to change a “murine” antibody to a “human” antibody, without altering the binding specificity of the antibody (U.S Pat. No. 5,225,539, 5,530,101, 6,331,415, all incorporated in their entirety).

Once suitable antibodies have been obtained, they may be isolated or purified by many techniques well known to those of ordinary skill in the art (see Antibodies: A Laboratory Manual, Harlow and Lane (eds.), Cold Spring Harbor Laboratory Press, 1988). Suitable techniques include peptide or protein affinity columns, HPLC or RP-HPLC, purification on protein A or protein G columns, or any combination of these techniques.

3. Uses of Antibodies

The antibodies may also be used to identify mimotopes of tau, including mimotopes of aggregated tau, which could then be used in a vaccine. A mimotope is a macromolecule, often a peptide, which mimics the structure of an epitope. Because of this property it causes an antibody response similar to the one elicited by the epitope. Briefly, the anti-tau antibodies are used to query a phage display library or other type of peptide library for a peptide that mimics the conformational antigenic determinant in aggregated repeat domain of tau. Such a peptide is a mimotope. The mimotope can be formulated and used as a therapeutic for tauopathies. For a vaccine, a mimotope that is antigenic, but not immunogenic, can be coupled to a carrier. Mimotopes can also be used for generation of antibodies, including polyclonal and monoclonal antibodies and scFv, Fab, recombinant antibodies, chimeric antibodies and the like, that will inhibit aggregation of tau and have a therapeutic effect.

Anti-tau antibodies, including scFv, Fab, Fab′ or F(ab)′, can be used to raise anti-idiotype antibodies, which are then used as a vaccine. Anti-idiotypic antibody may mimic the original antigen. In one embodiment, anti-idiotype antibodies are monoclonal or scFv, Fab, Fab′, F(ab)′ and may be generated by recombinant DNA techniques that are well-known in the art.

Antibodies, especially monoclonal antibodies, scFv, an F(ab') fragment, an F(ab) fragment and an F(ab′)₂ fragment, may be coupled to a protein transduction domain (PTD) or other molecule that can facilitate the crossing of the blood brain barrier. Two general classes of PTDs have been described, including positively charged transduction domains (cationic) and protein leader sequence derived domains (hydrophobic). Both are able to transduce wide variety of cell types. The cationic type domain is generally 10 to 30 amino acid residues in length and enriched in basic amino acids, e.g., arginine and lysine. Many PTDs are well-know and have been identified in proteins, such as synB peptide derived from protegrin, Drosophila homeodomain transcription factors antennapedia, HIV-1 transactivating protein TAT, engineered chimeric PTDs. In addition, PTDs may be identified by phage display methods. Sequences of exemplary PTDs include YGRKKRRQRRR (SEQ ID No.8) from HIV tat, MIIYRDLISH (SEQ ID No.9) from human translationally controlled tumor protein (TCTP), RQIKIWFQNRRMKW (SEQ ID No.10) from antennapedia, KLALKLALKALKAALKLA (SEQ ID NO.11), GWTLNSAGYLLGKINLKALAALAKKIL from galanin (SEQ ID NO. 12), GALFLGWLGAAGSTMGAWSQPKKKRKV (SEQ ID NO. 13) from SV40, RGGRLSYSRRRFSTSTGR (SEQ ID NO. 14) and RRLSYSRRRF (SEQ ID NO. 15) from protegrin. Antibodies may be coupled to a PTD as a fusion protein or chemically, using one of well-known methods.

4. Construction of DNA Compositions

When antibodies are to be delivered as a DNA composition, the composition will typically be an expression vector. In some embodiments, the vector is capable of autonomous replication. In other embodiments, the vector is a viral vector, insect vector, or a bacterial vector. The vector can alternatively be a plasmid, a phage, a cosmid, a mini-chromosome, a virus like particle (VLP), or a virus. Nucleic acid molecules can also be delivered in liposomes or adhered to nanoparticles. Materials and methodology for preparing liposomes and nanoparticles are well-known in the art. The sequence encoding an antibody is operatively linked to a promoter that is active in host cells. There will typically also be a polyA signal sequence, one or more introns, and optionally other control sequences, such as an enhancer. The promoter can be a constitutive promoter, an inducible promoter, or cell-type specific promoter. Such promoters are well known in the art.

The sequence of an antibody is readily determined for a monoclonal antibody. A variety of techniques can be used to clone, identify and sequence the antibody chains. In one technique, primers for the variable regions are used in an amplification reaction. The resulting amplified fragment can be inserted into a vector and grown or sequenced directly. With the sequence of the variable regions of the heavy and light chains, expression vectors can be constructed for scFv, Fv, Fab, and the like.

Also disclosed herein is a method of making a composition disclosed herein, comprising: obtaining sequence data representing the sequence of the composition; and synthesizing the composition. Tau peptides may be synthesized using automated peptide synthesizers, which are commercially available, and many companies provide synthesis services (e.g., American Peptide Company, Invitrogen). Following synthesis, rresulting antibodies may be used without further purification or purified, typically by HPLC, although alternative purification methods such as ion exchange chromatography and gel filtration chromatography may be used. Acceptable purity is at least 90% or at least 95% or at least 98% as assessed by analytical HPLC.

5. Formulations and Delivery of Antibodies

Anti-tau antibodies, including scFv, Fab fragment, Fab′2, may be formulated as a pharmaceutical composition for delivery to a subject (e.g., for use as a passive antibody therapy) or for the generation of an active vaccine to raise anti-idiotypic antibodies mimicking the antigenic determinant of tau. The compositions may include adjuvants and pharmaceutically acceptable excipients.

Anti-tau antibody compositions may comprise a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. intravenous, cutaneous or subcutaneous, nasal, intramuscular, intraperitoneal routes.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and other additives can be included, as required.

Immunogenic compositions comprise one or more anti-tau antibodies and hybrid antibodies (one antigenic determinant of antibody will bind one epitope and another one will bind another epitope).

The compositions may be packaged as a solution, but can also be packaged in dry form (e.g., desiccated), in which case, a user adds any necessary liquid. Typically, additives such as buffers, stabilizers, preservatives, excipients, carriers, and other non-active ingredients will also be present in the package. Additives are typically pharmaceutically acceptable and bio-compatible.

When antibodies are to be delivered as a DNA composition, the composition will typically be an expression vector. In some embodiments, the vector is capable of autonomous replication. In other embodiments, the vector is a viral vector, insect vector, or a bacterial vector. The vector can alternatively be a plasmid, a phage, a cosmid, a mini-chromosome, a virus like particle (VLP), or a virus. The delivery vehicle can also be a liposome or nanoparticle. The sequence encoding an antibody or will be operatively linked to a promoter that is active in host cells. There will typically also be a polyA signal sequence, one or more introns, and optionally other control sequences, such as an enhancer. The promoter can be a constitutive promoter, an inducible promoter, or cell-type specific promoter. Such promoters are well known in the art.

In addition, the protein or nucleic acid may be presented in separate containers or combined in a single container. A container can be a vial, ampoule, tube, or well of a multi-well device, reservoir, syringe or any other kind of container. The container or containers may be provided as a kit. If one or more of the containers comprises desiccated ingredients the liquids for reconstitution may be provided in the kit as well or provided by the user. The amount of solution in each container or that is added to each container is commensurate with the route of administration and how many doses are in each container. The compositions are generally provided sterile. Typical sterilization methods include filtration, irradiation and gas treatment.

Methods for treatment of tau neuropathies and related diseases are provided. Methods include administering a therapeutically effective amount of a composition disclosed herein. Administration is preferably in a “therapeutically effective amount” or “prophylactically effective amount”(as the case can be, although prophylaxis can be considered therapy), this being sufficient to show benefit to the individual. Effectiveness may be measured by any number of parameters or endpoints, such as an improvement in cognitive ability, a slowing down of cognitive decline, an improvement of physical abilities or slowing of physical decline, and the like. After the demise of a patient, the amount of neurofibrillary tangles and other symptoms of AD can be directly assessed and used to help guide determination of effective doses. The actual amount administered, and rate and time-course of administration, will depend on the nature and severity of protein aggregation disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980. Furthermore, a composition can be administered alone or in combination with other treatments, either simultaneously or sequentially dependent upon the condition to be treated.

The following examples are offered by way of illustration, and not by way of limitation.

### EXAMPLES

### Example 1

### Generation of Anti-Tau Antibodies to N-Terminal Region of Tau

In this example, an epitope of tau is used to generate antibodies specific to the N-terminal region. Immunogenicity of other tau peptides has been tested by epitope mapping.

The peptide from residue 2 through 18 of tau was fused with a foreign promiscuous Th (helper T cell) fragment of tetanus toxin TT (P30) and used to immunize mice. This region of tau is normally hidden when normally folded, but becomes exposed during aggregation of tau. The P30 epitope activates CD4+ T cells in H2^{bxs} and H2^{b} (data for production of IFN-γ and IL-4 not shown) mice as well as activates human helper T cells expressing various MHC class II/DR molecules.

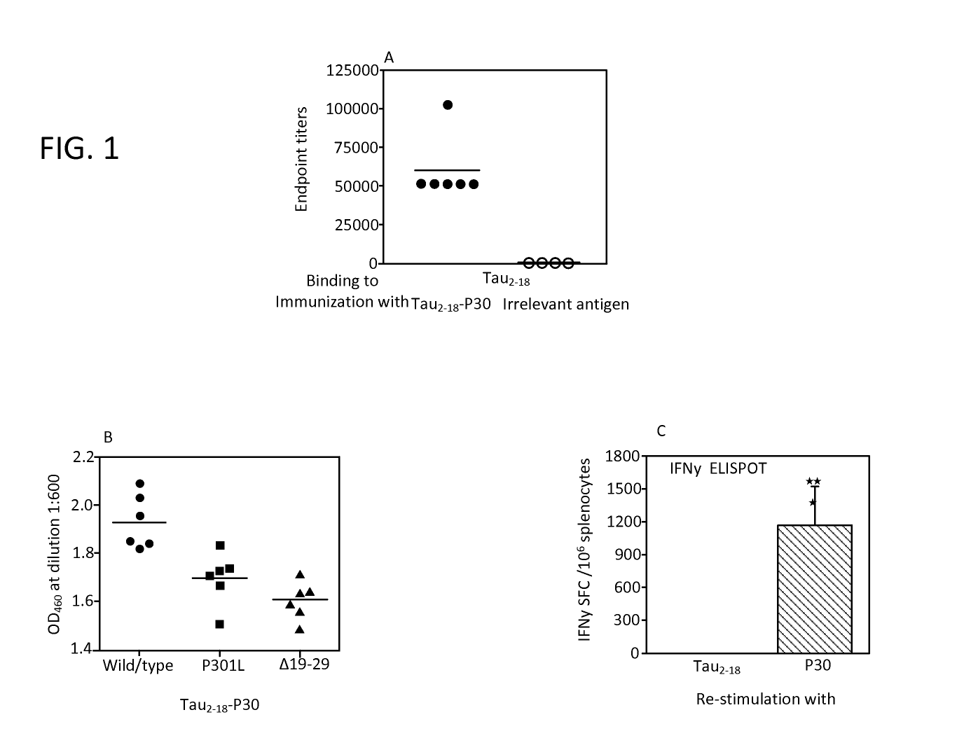

B6SJL mice (H2^{bxs} immune haplotype) were immunized with a tau₂₋₁₈-P30 immunogen formulated in the adjuvant Quil A (also known as QS21). Both humoral (ELISA) and cellular (ELISPOT) immune responses were measured. Immunization induced high titers of tau₂₋₁₈-specific antibodies that also recognized 4R/0N wild-type tau, 4R/0N tau with a P301 L alteration, and 4R/0N tau with a deletion of residues 19-29 (FIGS. 1A, B). The epitope vaccine also induced a strong T cell response that was specific to P30, but not tau₂₋₁₈ (FIG. 1C). In conclusion, tau₂₋₁₈-P30 vaccine in QuilA adjuvant produced antibodies specific to various tau proteins and these antibodies can be used for passive vaccination of subjects at various stages of tauopathy (e.g. Alzheimer's disease).

### Example 2

### Generation of Anti-Tau Antibodies to C-Terminal Region of Tau

In this example, another epitope of tau is used to generate antibodies specific to the C-terminal region.

The peptide from residue 382 through 418 of tau was fused with a synthetic foreign promiscuous Th fragment, PADRE (comprising the sequence AKFVAAWTLKAAA (SEQ ID No. 16) and used to immunize mice. This region of tau is located at the C-terminus of the molecule and is normally buried inside the molecule, but becomes exposed during aggregation of tau prior to truncation. PADRE is an example of a synthetic sequence that serves as an epitope which activates CD4+ T cells in mammals (Alexander et al. Immunity 1:751, 1994).

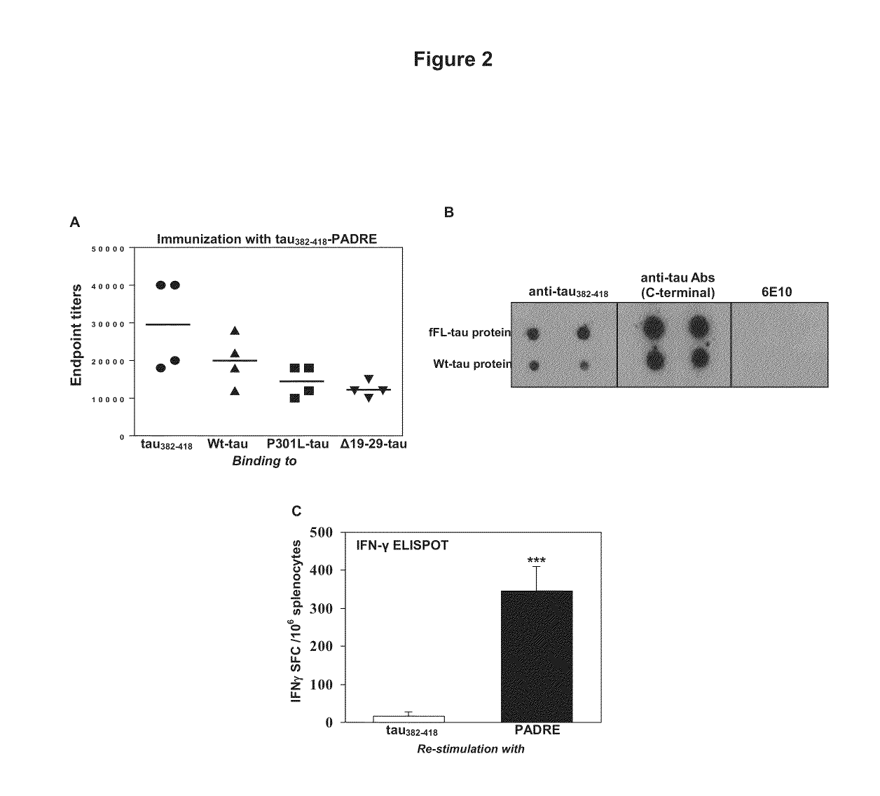

Fisher rats were immunized with a tau₃₈₂₋₄₁₈-PADRE immunogen formulated in the adjuvant Quil A (also known as QS21). Both humoral (ELISA and Dot Blot assay) and cellular (ELISPOT assay) immune responses were measured (FIG. 2). Immunization induced anti-tau₃₈₂₋₄₁₈-specific antibodies that also recognized various tau molecules (FIG. 2A) and fibrillar tau molecules (FIG. 2B). The epitope vaccine also induced T cell response that was specific to PADRE, but not tau₃₈₂₋₄₁₈ (FIG. 2C). In conclusion, tau₃₈₂₋₄₁₈-PADRE immunogen in QuilA adjuvant produced antibodies specific to various tau proteins and these antibodies can be used for passive vaccination of subjects with various stages of tauopathy.

### Example 3

### Therapeutic Potency of Anti-Tau Antibody

This example shows the effects of administering anti-tau antibodies on inhibiting aggregation of tau.

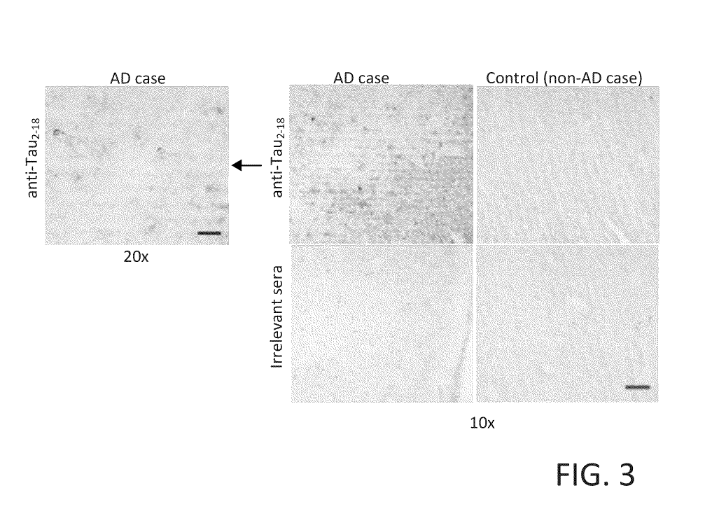

Sera from experimental mice and ratsimmunized with the epitope vaccine and control animals immunized with an irrelevant antigen were tested on brain sections from AD and non-AD cases. FIG. 3 shows that immune sera at a dilution 1:500 from experimental, but not control, mice (FIG. 3A) and rats (FIG. 3B) recognized neurofibrillary tangles (NFT) in brain from an AD patient (Tangle stage V, Plaque stage C). Importantly, the same immune sera did not bind tau in the brain sections from non-AD case. Thus, antibodies generated after immunization of tau epitope vaccines are specific to the only pathological form of tau.

Moreover, anti-tau₂₋₁₈ antibodies purified from the sera of vaccinated mice were tested for their ability to inhibit cell-to-cell propagation of tau aggregates, using the method of Kfoury et al. (Kfoury, N. et al. J Biol Chem 287, 19440-19451 (2012)). The antibodies were able to inhibit cell-cell progagation of both full-length tau and repeat domain (RD) aggregates, evidencing the therapeutic benefit of these antibodies.

More specifically, trans-cellular movement of aggregated tau was assessed in HEK293 cells transfected with tau repeat domain (RD) containing a deletion of lysine at position 280 (ΔK280) and fused to cyan or yellow fluorescent protein (RD-CFP) or (RD-YFP) (ΔK-C):(ΔK-Y). A second population of HEK293 cells was transfected with hemagglutinin-tagged tau (RD) containing two disease-associated mutations that increase aggregation: P301 L and V337M (LM) (LM-HA). When the two cell populations were co-cultured, trans-cellular propagation of LM-HA aggregates from donor cells (HEK293 cells transfected with LM-HA) induces aggregation of ΔK-C:ΔK-Y in recipient cells (HEK293 transfected with RD-CFP/RD-YFP) as detected by fluorescence resonance energy transfer (FRET) between CFP and YFP, which yields a signal detected by a fluorescence plate reader. If anti-tau antibodies are added to this system and block propagation of tau, then FRET is inhibited.

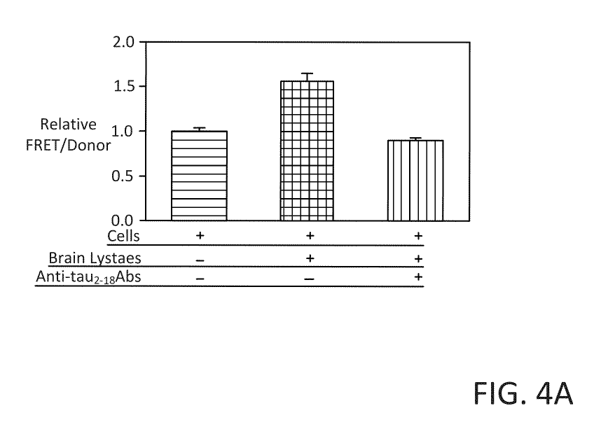

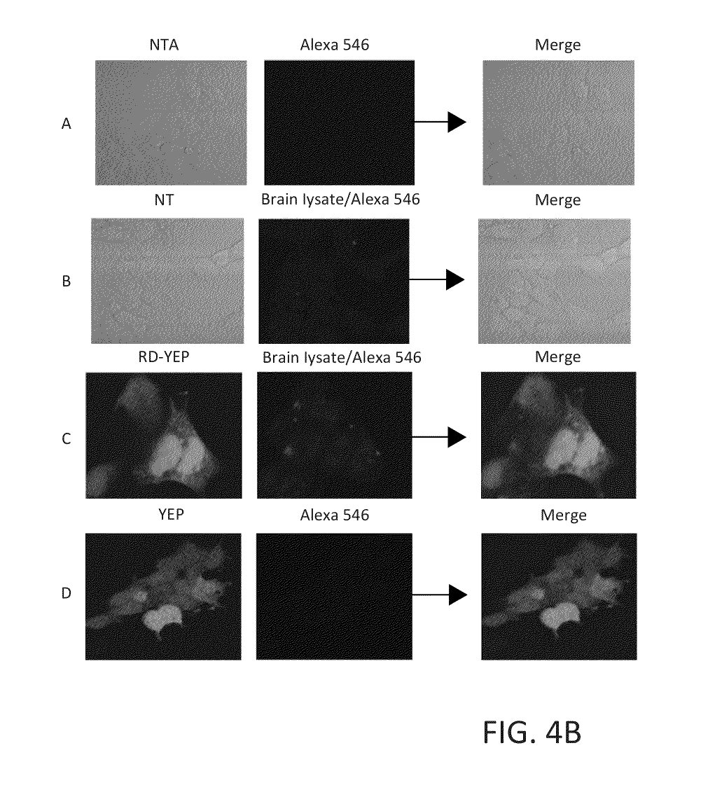

In another set of experiments, the ability of anti-tau₂₋₁₈ antibody was tested for ability to block full-length tau aggregates from entering a cell and inducing aggregation of intracellular RD. The experiment was designed as described above except that the aggregation ΔK-C:ΔK-Y in recipient cells was induced by adding aggregated tau from brain lysates of P301S Tg (transgenic) mice that were either untreated or pre-incubated with anti-tau₂₋₁₈ antibody. As shown in FIG. 4A, as expected addition of untreated brain lysate increased a FRET signal, whereas pre-treating of brain lysate with anti-tau₂₋₁₈ antibody completely blocked the ability of brain lysate to induce the aggregation of RD in recipient cells (FIG. 4A). Importantly, using confocal microscopy brain lysate/anti-tau₂₋₁₈ antibody complexes are shown to internalize into the RD-YFP transfected cells (FIG. 4D), but not in control mock-transfected cells (NT) with added brain lysates from Tg mice (FIG. 4C). Of course, antibodies were not detected in NT cells (FIG. 4B) or in RD-YEP cells when tau aggregates from Tg mice were not added to the test tubes (FIG. 4 E)

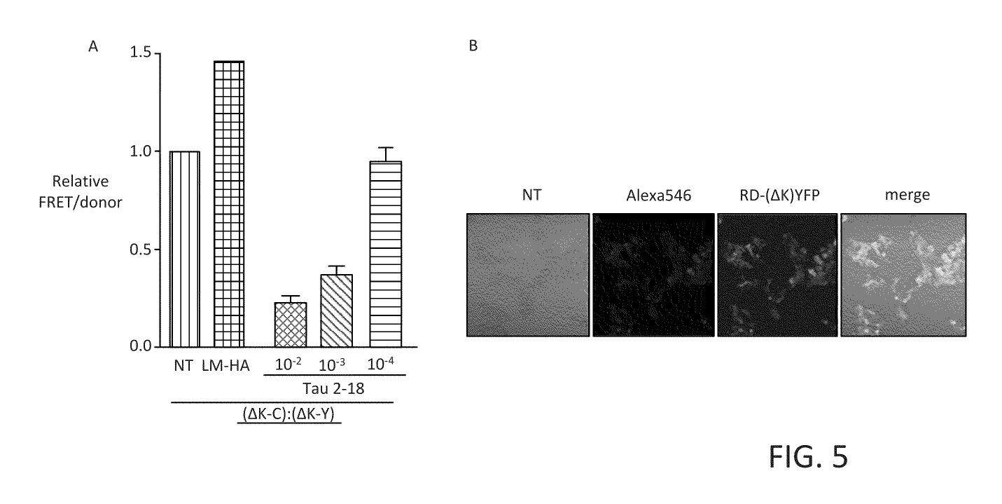

Unexpectedly, not only did anti-tau₃₈₂₋₄₁₈ antibodies specific to C-terminus, but anti-tau₂₋₁₈ antibodies specific to the N-terminus of tau also blocked inhibition of tau aggregation in the case when full-lengths of tau from the brain lysates of P301S Tg mice are not used (FIG. 5). To measure baseline endogenous aggregation, a (ΔK-C):(ΔK-Y) cell population was co-cultured with mock-transfected cells (NT). To test the ability of antibodies to block cell-to-cell transfer of RD aggregates, anti-tau₂₋₁₈ antibody was added to the culture at different dilutions (10⁻², 10⁻³ and 10⁻⁴) and incubated for 48 h. A dose-dependent reduction of FRET signal, indicating reduced transcellular propagation of LM-HA aggregates, is observed with both anti-tau₂₋₁₈ and anti-tau₃₈₂₋₄₁₈ antibodies (FIG. 5A), whereas nonspecific IgG had no effect (data not shown). Anti-tau₃₈₂₋₄₁₈ antibodies inhibit tau propagation to a greater degree than anti-tau₃₈₂₋₄₁₈ antibodies (FIG. 5A). Because the tau₂₋₁₈ peptide is localized outside of the RD region, these data indicate that anti-tau₂₋₁₈ antibody recognizes a conformational antigenic determinant (mimotope(s) in aggregated RD and blocks its cell-to-cell propagation. Furthermore, using confocal microscopy anti-tau₂₋₁₈ antibody (detected by secondary anti-mouse Ig labeled by Alexa546) is detected inside the cells with aggregated RD tau (FIG. 5B). Thus when RD(ΔK) aggregates were added instead of brain lysates, binding of anti-tau₂₋₁₈ antibodies was observed to RD(ΔK)YFP aggregates and internalization of antibody/RD(ΔK)YFP complexes. In other words, −tau₂₋₁₈antibodies enter the cells after binding to aggregated tau outside of the cells.

Importantly, when RD(ΔK) was replaced with a mutant form of tau containing two proline substitutions, I227P and I308P (termed PP), which inhibit β-sheet formation and fibrillization, no complexes were formed and no internalization of antibodies was observed (data not shown). These data confirm that anti-tau₂₋₁₈ antibody recognizes a conformational antigenic determinant (mimotope/s) in aggregated RD.

These examples show that therapeutic anti-tau antibodies can be generated with a non-phosphorylated tau molecules or their derivatives (e.g. B cell epitopes). Indeed, non-phosphorylated tau may be used for generation of therapeutic antibodies that will be safe to administrate to subjects with tauopathy, because such antibodies will not get inside normal cells and inhibit function of normal tau molecules.

### Example 4

### Other Therapeutic Antibodies Specific to Various Regions of Tau

To map other candidate epitopes of non-phosphorylated tau regions for the generation of therapeutic antibodies, antisera were obtained from tau transgenic mice (rTg4510; SantaCruz et al. Science 309:476, 2005) immunized with full length tau (0N4R; SEQ ID No. 2). ELISA methodology was used to detect binding of polyclonal sera to recombinant tau proteins from 1 aa to 50 aa, from 50 aa to 100aa, from 100aa to 150aa; from 150aa to 200aa, from 200aa to 250aa; from 250aa to 300aa; from 300aa to 350aa; from 350aa to 400aa; from 400aa to 441aa; thus the entire sequence of 0N4R molecule was assessed. (Overlapping peptides could also be used.) Briefly, wells of 96-well plates (Immulon II, Dynatech) were coated with 2.5 μM of appropriate recombinant protein (pH 9.7), and sera from experimental and control Tg mice were added in duplicate at dilution of 1:500. Anti-mouse IgG conjugated with horseradish peroxidase (HRP) was added, and plates were analyzed spectrophotometrically at 405 nm. Data demonstrated that anti-tau antibodies bind strongly to regions spanning aa 1 to 50 of tau protein and do not bind aa 250-300 at all (Table 1). Moderate binding was detected in wells coated with recombinant tau proteins spanning aa 150 to 200, 200 to 250; 350 to 400; and 400-441. Finally low binding was detected in wells coated with recombinant tau proteins spanning aa 100 to 150 and 300-350. These data demonstrate regions of tau that can be used to produce therapeutic antibodies for therapeutic benefit by passive vaccination (immunization) of subjects with tauopathy.

TABLE 1

| | No. of aa (SEQ | Strength of |
|---|---|---|
| Protein name | ID NO. 5) | binding |
| Protein 1 | 1-50 | ++++ |
| Protein 2 | 50-100 | − |
| Protein 3 | 100-150 | + |
| Protein 4 | 150-200 | ++ |
| Protein 5 | 200-250 | ++ |
| Protein 6 | 250-300 | − |
| Protein 7 | 300-350 | + |
| Protein 8 | 350-400 | ++ |
| Protein 9 | 400-425 | ++ |

From the foregoing, it will be appreciated that, although specific embodiments have been described herein for purposes of illustration, various modifications may be made without deviating from the spirit and scope of the invention. Accordingly, the invention is not limited except as by the appended claims.

We claim:

1. An antibody that binds to an N-terminal region of tau or a C-terminal region of tau.

2. The antibody of claim 1, wherein the antibody binds to tau₂₋₁₈ (SEQ ID NO. 7).

3. The antibody of claim 2, wherein the antibody binds to tau₃₈₂₋₄₁₈ (SEQ ID No. 17).

4. The antibody of claim 1, wherein the antibody is capable of binding to pathologically aggregated tau.

5. The antibody of claim 1, wherein the antibody is capable of binding to aggregated repeat domains of tau.

6. The antibody of claim 1, wherein the antibody binds to each of the six isoforms of tau.

7. The antibody of claim 1, wherein the antibody is capable of binding a mimotope of tau.

8. The antibody of claim 1, wherein the antibody is a monoclonal antibody.

9. The antibody of claim 8, wherein the antibody is a humanized antibody.

10. The antibody of claim 1, wherein the antibody is a single-chain Fv fragment, an F(ab′) fragment, an F(ab) fragment or an F(ab′)2 fragment.

11. The antibody of claim 10, wherein the single-chain Fv fragment, the F(ab′) fragment, the F(ab) fragment or the F(ab′)2 fragment is conjugated with a protein transduction domain (PTD).

12. The antibody of claim 1, wherein the antibody is a chimeric antibody.

13. The antibody of claim 1, wherein the antibody is capable of inhibiting the cell to cell propagation of pathological, aggregated tau.

14. A nucleic acid molecule that encodes an antibody that binds an N-terminal region of tau or a C-terminal region of tau.

15. A hybridoma producing an antibody that binds to an N-terminal region of tau or a C-terminal region of tau.

16. A pharmaceutical composition comprising an antibody that binds to an N-terminal region of tau or a C-terminal region of tau and a pharmaceutically acceptable excipient.

17. A pharmaceutical composition comprising a nucleic acid molecule that encodes an antibody that binds an N-terminal region of tau or a C-terminal region of tau.

18. A method of preventing or treating a tauopathy in a mammal, comprising administering to a mammal in need a therapeutically effective dose of an antibody that binds an N-terminal region of tau or a nucleic acid molecule that encodes an antibody that binds an N-terminal region of tau.

19. The method of claim 18, wherein the prevention or treatment inhibits or slows down formation of tau aggregates in the brain of a mammal.

20. The method of claim 18, wherein the prevention or treatment inhibits or slows down formation of neurofibrillary tangles in the brain of the mammal.

21. The method of claim 18, wherein the prevention or treatment inhibits polymerization of tau.

22. A method of preventing or treating a tauopathy in a mammal, comprising expressing in the brain of a mammal a nucleic acid molecule that encodes an antibody that binds an N-terminal region of tau.

23. A non-phosphorylated N-terminal region of tau.

24. The tau of claim 22, wherein the region comprises amino acids 2-18.

25. The tau of claim 22, wherein the tau is coupled to a carrier molecule.

26. A method of raising an immune response to tau, by administering to an animal a composition comprising a non-phosphorylated region of tau coupled to a carrier molecule. 27 An antibody to tau, wherein the antibody binds to residues 100-150 or residues 150-200 or residues 200-250 or residues 300-350 or residues 350-400.
